(19) United States
(12) Patent Application Publication
(10) Pub. No.: US 2022/0296536 A1
(43) Pub. Date: Sep. 22, 2022

(54) PAIN MANAGEMENT METHODOLOGY

(71) Applicant: David FLINT, Darwin (AU)

(72) Inventor: David FLINT, Darwin (AU)

(21) Appl. No.: 17/637,293

(22) PCT Filed: Aug. 24, 2020
(86) PCT No.: PCT/AU2020/000086

§ 371 Date: Feb. 22, 2022

(30) Foreign Application Priority Data
Aug. 23, 2019 (AU) 2019903081

(51) Int. Cl. A61K 31/05; A61P 25/04; H04R 1/10

(52) U.S. Cl. CPC A61K 31/05; A61P 25/04; H04R 1/1083; H04R 1/1008; H04R 1/1041

(57) ABSTRACT

A pain management system and methodology for preventing or alleviating chronic pain, and/or pain associated with a medical procedure prior to the medical procedure being performed, includes (a) aurally isolating a patient for a chosen period shortly prior to the patient receiving propofol in accordance with step (c), (b) exposing the patient to a repetitive, positive message during the chosen period, and (c) subsequently administering an effective quantity of propofol to the patient. For step (b), the repetitive, positive message may be delivered from a pre-recorded memory card inserted in headphones.

### CROSS-REFERENCE TO RELATED APPLICATIONS

The present application is a Section 371 of International Application No. PCT AU2020/000086, filed Aug. 24, 2020, which was published as International Patent Application Publication No. 2021/035276 and titled, “Pain Management Methodology,” which claims priority to Australian Patent Application No. 2019903081, filed Aug. 23, 2019, the disclosures of which are incorporated herein by reference in their entireties.

### TECHNICAL FIELD

The present invention broadly relates to a method of preventing or alleviating pain in a patient. In particular, this invention is concerned with a method of alleviating chronic pain, and/or pain associated with a medical procedure prior to the medical procedure being performed.

### BACKGROUND OF THE INVENTION

Pain management in humans has long been the focus of a significant amount of research.

Chronic pain may be addressed in various ways, including by analgesics (non-opioids), opioids and surgery.

Post-operative pain can delay recovery. It is a major concern for patients and health professionals. Post-operative pain may prolong healing and delay discharge. If not adequately managed, post-operative pain may become chronic pain. Analgesics or opioids are usually used for post-operative pain.

Opiate analgesics, administered intravenously, are regarded as important drugs for pain, but can delay post-operative recovery. They may also depress respiration, to the extent of requiring mechanical ventilation of the patient.

There can be other problems with current pain management. For some patients, analgesics can cause nausea. For severe pain, sometimes analgesics are ineffective. Opioids can induce addiction. The cost of analgesics and opioids is also a significant factor.

Attempts have been made to improve management of pain, especially post-operative pain, by focusing on the use of a wider range of drugs and various routes of administration. As well as non-opioid analgesics and opioids, other drugs such as morphine, fentanyl, meperidine and ketamine have been used. Administrative routes have included intravenous, oral, transdermal and epidural. However, there is still a need for improvement in pain management.

It is therefore an object of the present invention to provide a pain management methodology which is capable of greatly improved pain management

The present invention is based on the discovery that certain pain, including post-operative pain, may be alleviated or eliminated by following a protocol which, in the case of a medical operation or surgery, commences pre-surgery and involves the use of propofol prior to surgery.

### BRIEF SUMMARY OF THE INVENTION

Accordingly, in one aspect, the present invention provides a method for preventing or ameliorating pain in a patient, where the method including the steps of:
(a) aurally isolating the patient for a chosen period shortly prior to the patient receiving propofol in accordance with step (c);
(b) exposing the patient to a repetitive, positive message during the chosen period; and
(c) subsequently administering an effective quantity of propofol to the patient.

In a second aspect, the invention provides propofol when used in a method for preventing or ameliorating pain in a patient, the method including the steps of:
(a) aurally isolating the patient for a chosen period shortly prior to the patient receiving propofol in accordance with step (c);
(b) exposing the patient to a repetitive, positive message during the chosen period; and
(c) subsequently administering an effective quantity of propofol to the patient.

In a third aspect, the invention provides a set of headphones when used in the method of the first aspect, wherein the message is pre-recorded on a suitable medium for playing through the headphones to the patient.

The pain to be addressed may be post-operative pain following surgery undertaken after the administration of propofol. In addition, extreme chronic pain in a patient not responding to opioids may be addressed by the method of the invention including administration of propofol.

The method of the invention, requiring the use of propofol, may be applicable for use with any type of surgery. The method of the invention does not require that surgery follows the administration of propofol.

The term ‘aural isolation’ means that the patient is fully or substantially isolated from ambient sound and from being addressed by any third party. The exception to this is where the repetitive, positive message is being delivered by a third party (‘deliverer’) rather than via a recording, for example. In that case, only the deliverer should be present with the patient during aural isolation.

The patient may be aurally isolated by any suitable means. Where the repetitive, positive message is delivered by a deliverer, aural isolation is best effected by the patent being isolated in a quiet room with only the deliverer being present. In this embodiment, the deliverer preferably sits beside the patent and repeats the message to the patient in a calm, clear voice, during all or a substantial part of the chosen period.

In another preferred embodiment, a set of headphones is used to aurally isolate the patient in a quiet room and to deliver the repetitive, positive message. Preferably, the headphones are circumaural so as to surround the patient's ears, resembling ear muffs. The headphones may include noise cancelling properties, as is known in the art.

The headphones in this embodiment may include a disposable interface between the headphone or muff for each ear and the patient, for hygienic purposes and to avoid contamination. The interface may be made of any suitable material. Preferably, the interface if used is made of soft, non-woven material such as that used for disposable theatre gowns and surgical drapes. An example is a bonded polyolefin material known as SMS, have a layer of spunbonded material (‘S’) bonded on either side of a layer of meltblown material (‘M’), as described in U.S. Pat. No. 4,041,203, the contents of which are incorporated herein by reference.

The headphones are preferably sterilised or wiped down with antiseptic before being used on each patient.

The patient may be aurally isolated for any suitable chosen period. In a preferred embodiment, the patient is aurally isolated for about 30 minutes. In this embodiment, aural isolation commences after the patient has received any pre-surgery treatment, such as light sedation to reduce anxiety, and concludes just before the patient is wheeled into an operating theatre or has an intravenous catheter inserted for administration of anaesthetic. The patient may be in a quiet room or in an operating theatre vestibule during the chosen period of aural isolation. Preferably, the patient is prone during aural isolation. One or more walls of the room or vestibule may have a poster, sign board or the like, explaining the method of the invention and emphasizing the need for aural isolation.

It is especially preferred that the patient is not distracted by visual stimuli during the aural isolation period. Hence there should be no third party present, except for the deliverer (if the message is to be delivered in person), the attending nurse and/or anesthetist. Other distractions should be removed or minimised.

The patient is exposed to the repetitive, positive message during the chosen period. The message may be pre-recorded on a suitable medium, which may be analogue but which preferably is digital. Recording may be effected by any suitable means as known in the art. The message may be stored on any suitable medium, preferably a digital medium such as a memory card. The medium is preferably kept secure, as further discussed below.

In a preferred embodiment where aural isolation is effected by using headphones, the message may be delivered to the patient via the headphones. In this embodiment, the message may be recorded on a medium stored in the headphones themselves, either permanently or on a memory card, receivable in a slot in the headphones.

An advantage of using headphones to deliver the message is that the message may be translated into any desired language.

For security, in an embodiment, the headphones are secured in a suitable locker, cabinet or other suitable container when not in use. Preferably, a locker is used which also provides a charging facility for the headphones if they have a rechargeable battery. In this embodiment, the headphones may be locked in the locker when not in use, with a light or other indicia on the outside of the locker indicating when the headphones inside are fully charged. This feature can be useful in a hospital or other large facility having a plurality of the headphones of the invention stored in an array of lockers.

The repetitive, positive message may take any suitable format. The message is preferably short and clear and is repeated for all or substantially all of the chosen period. Examples of the message are:

‘When I wake up, I will have no pain’; or

‘When I wake up I will be pain free’;

In the method of the invention, propofol is administered to the patient. Propofol (2,6-diisopropylphenol) is known and is sold in some markets under the commercial name Diprivan. Propofol may be formulated in a 1% solution, providing 10 mg of drug in an aqueous solution of 10% soybean oil, 2.25% glycerol, and 1.2% egg phosphatide

In the past, propofol has been used as an agent for induction and maintenance of anaesthesia. Propofol has a rapid onset and a short duration of action. It has minimal side effects. It has the advantages of the patient rapidly regaining consciousness after surgery, with minimal residual central nervous system effects. It also has significant amnesia effects and can reduce post-operative nausea and vomiting.

The effective quantity of propofol dose for the patient can be ascertained by an anesthetist or other suitable person, taking into account the body weight of the patient and other relevant factors, such as the age of the patient.

The method of the invention may be followed by the application of a general anaesthetic and surgery in the normal way. However, the method of the invention may also alleviate pain in the absence of surgery: surgery is not a required step in the method of the invention.

Propofol is preferably administered intravenously by use of a syringe.

While the physiological and/or psychological reason for success of the method of the invention has not been ascertained, it is believed that the aural isolation, repetitive positive message and use of propofol in the method of the invention may combine to cause the patient to be substantially or totally pain-free after use of the method. It is also believed that the amnesia effects of propofol may play a role.

Best Methods of Carrying out the Invention

The invention will now be described in connection with certain non-limiting examples thereof.

In each of the examples, below, the method of the invention was used: the patient was aurally isolated in a quiet room, being the waiting room or vestibule to an operating theatre with only the deliverer present during the aural isolation period. The patient was exposed to the repetitive, positive message delivered orally by the deliverer. The chosen period was between 20 and 30 minutes. The deliverer repeated the message constantly during the chosen period. The message was: ‘When I wake up, I will have no pain’.

At the conclusion of the chosen period, the patient was administered propofol and became unconscious.

None of the patients in the examples below required pharmaceutical pain relief after use of the method of the invention. All experienced accelerated recovery from surgery, when used.

### EXAMPLE 1

Patient A was a male having terminal prostate cancer. Patient A was suffering a high level of pain, for which pharmaceutical treatment, even morphine, had become ineffective.

The method of the invention was used on Patient A, prior to Patient A undergoing surgery to alleviate an incontinence problem. Patient A was free of pain from the time he became conscious in the recovery room after the surgery until three weeks later (when Patient A chose legal voluntary euthanasia).

### EXAMPLE 2

Patient B had bleeding on the brain at four sites. The method of the invention was followed prior to Patient B having his skull drilled to drain blood. Patient B experienced no pain.

### EXAMPLE 3

Patient B underwent cataract surgery using the method of the invention and experienced no pain post-surgery.

### EXAMPLE 4

Patient B had radical endoscopic sinus surgery. The method of the invention was used. Patient B experienced no post-operative pain.

### EXAMPLE 5

Patient B had a hip replacement and had no pain post-surgery following use of the method of the invention. In addition, Patient B experienced accelerated recovery. Patient B was ambulatory within one hour, instead of requiring 7 days bed rest as normally expected.

### EXAMPLE 6

Patient B had a painful infection within his left shoulder. X-rays revealed a secondary infection. After use of the method of the invention and surgery, Patient B was pain-free.

### EXAMPLE 7

Patient B had osteomyelitis (infection of the bone) in his right hip, causing damage to veins in his leg. The method of the invention was used prior to surgery involving stripping of the veins and a skin graft. Patient B had no pain post-surgery.

### EXAMPLE 8

Patient B had open-heart surgery to replace an aortic valve. Because of complications, the open-heart surgery was repeated two more times. The method of the invention was used for each of the three surgeries. Patient B had no pain post-surgery each time.

### EXAMPLE 9

Patient C had a hip replacement. The method of the invention was used. Patient C had no pain post-surgery.

### EXAMPLE 10

Patient D had her uterus removes in a radical hysterectomy. The method of the invention was used. Patient D experienced no pain post-surgery.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown. In the drawings:

FIG. 1 is a side elevational view of an embodiment of a set of headphones suitable for use in connection with the invention;

FIG. 2 is a front elevational view of the headphones of FIG. 1;

FIG. 3 is an enlarged side elevational view of the headphones of FIG. 1 and FIG. 2, with a detailed view of the location of a memory card slot taken from within the dashed circle of FIG. 3;

FIG. 4 is a top plan views of an embodiment of a pair of black disposable interfaces for mounting on a headphone, one of the pair being shown from the inside and the other of the pair being shown from the outside; and

FIG. 5 shows a front perspective view of white disposable interfaces the same as those in FIG. 4 except for colour, mounted on a headphone similar to that in FIGS. 1 to 3.

It will be appreciated that the drawings are not all on the same scale.

### DETAILED DESCRIPTION OF THE INVENTION

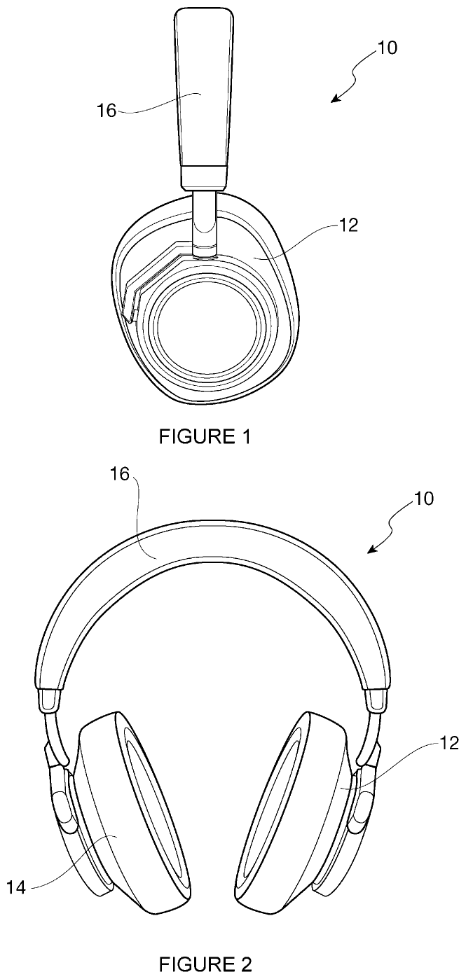

The headphones shown in each of FIGS. 1 to 3 are commercially available under the name ‘Bluedio-T7+’ from Cellway, Floor 2, Building A, No. 46 Pupai Road, Ailian Town, Longgang District, Shenzhen China. These headphones also have noise-cancelling functionality, which can be desirable in relation to the invention.

Referring first to FIGS. 1 and 2, headphones 10 are circumaural in that they are designed to fit over and around the ears of a patient. Headphones 10 have first headphone 12 and second headphone 14, linked by headband 16. Headphone 12 includes controls including for volume and activating noise cancelling function.

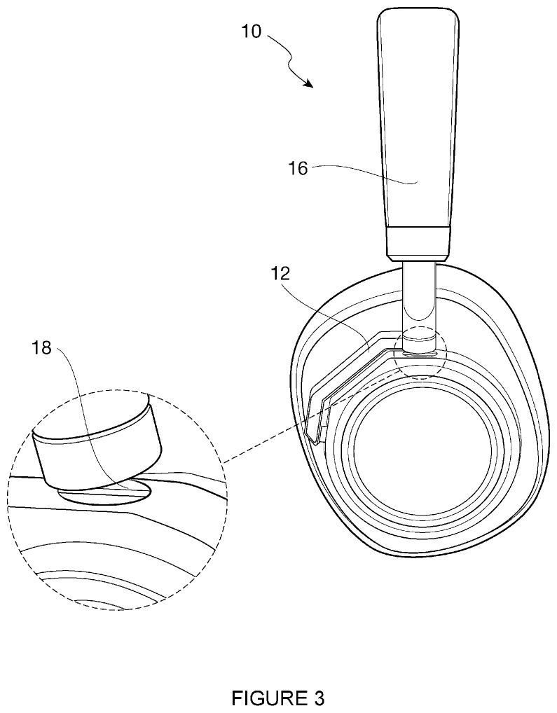

Memory card slot 18 is also located on the outside of headphone 12 (refer FIG. 3, as enlarged in circled portion). Memory card slot 18 is adapted to receive an SD 32 G memory card. In this embodiment, the repetitive, positive message is pre-recorded on the memory card in the desired language and inserted in slot 18 prior to use in the method of the invention.

Headphones 10 have a 600 MAH polymer battery which is rechargeable via a USB type charging cable plugged into a charging port (not shown) located on the outside of headphone 12.

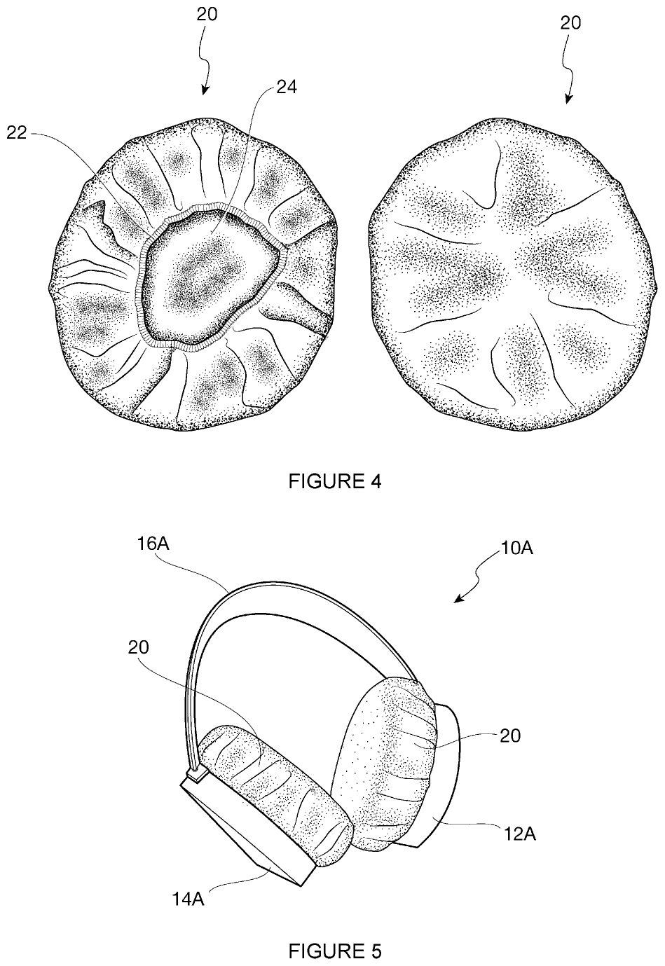

Disposable interface 20 in FIG. 4 is made of a nonwoven sanitary material such a SMS and is stretchable so as to fit over the inside surface of each of headphone 12A and headphone 14A of headphones 10A having band 16A, as shown in FIG. 5. Elastic 22 is arranged around the circumference of opening 24, to assist in retaining interface 20 on headphone 12A or 14A. Opening 24 is about 5 cm in diameter. A new interface 20 is mounted on headphone 12A and headphone 14A for each new patient and discarded after use.

The embodiments described above relate to preferred embodiments of the present invention only and are given by way of illustration. Changes, modifications and variations may be made without departing from the spirit and scope of the present invention.

Those skilled in the art will appreciate that the invention described herein is susceptible to variations and modifications other than those specifically described. All such variations and modifications are to be considered within the scope and spirit of the present invention the nature of which is to be determined from the foregoing description.

### INDUSTRIAL APPLICABILITY

Now that preferred embodiments of the present invention have been described, it will be apparent to a skilled person in the art that the method of the present invention may offer at least the following advantages:
1. Relief or elimination of post-operative pain, allowing accelerated recovery and healing as well as early discharge from hospital;
2. Avoiding post-operative pain becoming chronic pain;
3. Reduction or elimination of the need for pharmaceutical treatment of pain, reducing cost to the patient and removing danger of addiction; and
4. Enhancement of the patient's quality of life even if the patent has a terminal prognosis.

1. A method for preventing or ameliorating pain in a patient, the method including the steps of:
(a) aurally isolating the patient for a chosen period shortly prior to the patient receiving propofol in accordance with step (c);
(b) exposing the patient to a repetitive, positive message during the chosen period; and
(c) subsequently administering an effective quantity of propofol to the patient.

2. The method of claim 1, wherein the step of aurally isolating the patient includes using a set of headphones on the ears of the patient.

3. The method of claim 2, wherein the message is delivered to the patient through the headphones.

4. The method of claim 1, wherein the chosen period is about 20 to 30 minutes.

5. The method of claim 1, wherein the patient is exposed to the message during all or a substantial part of the chosen period.

6. The method of claim 1, wherein the message contains substantially the following words:
‘When I wake up, I will have no pain’; or
‘When I wake up I will be pain free’;
the message being repeated during the chosen period.

7. Propofol when used in a method for preventing or ameliorating pain in a patient, the method including the steps of:
(a) aurally isolating the patient for a chosen period shortly prior to the patient receiving propofol in accordance with step (c);
(b) exposing the patient to a repetitive, positive message during the chosen period; and
(c) subsequently administering an effective quantity of propofol to the patient.

8. A set of headphones when used in the method of claim 1, wherein the message is pre-recorded on a suitable medium for playing through the headphones to the patient.

9. The set of headphones of claim 8, having a pair of headphones, each being circumaural.

10. The set of headphones of claim 8, wherein the medium is a memory card adapted for insertion in a slot in one of the headphones.

11. The set of headphones of claim 9, wherein in use a disposable interface is disposed between each headphone and the patient.

12. The set of headphones of claim 11, wherein the interface includes a soft non-woven material.

13. The set of headphones of claim 12, wherein the soft non-woven material is made of a bonded polyolefin material having spunbond and/or meltblown layers.
